(19) United States
(12) Patent Application Publication
(10) Pub. No.: US 2024/0415500 A1
(43) Pub. Date: Dec. 19, 2024

(54) OVER THE SCOPE DEVICE AND METHOD FOR TREATING TISSUE USING A PATCH

(71) Applicant: BOSTON SCIENTIFIC MEDICAL DEVICE LIMITED, Galway (IE)

(72) Inventors: Sharath Kumar G, Kanakapura (IN); Deepak Kumar SHARMA, Muzaffarnagar (IN)

(21) Appl. No.: 18/672,942

(22) Filed: May 23, 2024

Related U.S. Application Data
(60) Provisional application No. 63/508,380, filed on Jun. 15, 2023.

(51) Int. Cl. A61B 17/00

(52) U.S. Cl. CPC A61B 17/0057; A61B 17/00234; A61B 2017/00238; A61B 2017/0034; A61B 2017/00367; A61B 2017/00592; A61B 2017/00597; A61B 2017/0061; A61B 2017/00623; A61B 2017/0065; A61B 2017/00676

(57) ABSTRACT

A device includes a cap, a patch deployment mechanism, and a therapeutic patch. The cap includes a wall defining a chamber with an open distal end. The mechanism is slidably received within the chamber in an insertion configuration and includes proximal and distal members coupled by bowing members. The bowing members expand radially outward relative to the insertion configuration when longitudinally compressed via movement of the distal member toward the proximal member. The patch is wrapped around the mechanism and is forced radially outward into contact with and adhere to a target portion of tissue when the mechanism is longitudinally compressed and the bowing members are radially expanded. The mechanism is to radially retracted after the patch has been adhered to the portion, from a deployed configuration to the insertion configuration to separate the patch from the mechanism leaving the patch in a desired position adhered to the portion.

### PRIORITY CLAIM

The present disclosure claims priority to U.S. Provisional Patent Application Ser. No. 63/508,380 filed Jun. 15, 2023; the disclosure of which is incorporated herewith by reference.

### FIELD

The present disclosure relates to a minimally invasive delivery of a patch to cover a large portion of target tissue to achieve, for example, hemostasis. More specifically, the present disclosure relates to the intraluminal delivery of a patch positioned over an expandable body so that, upon reaching the target site, the patch may be moved radially outward to contact, adhere to and cover a large area of target tissue to be treated.

### BACKGROUND

The treatment of conditions such as bleeding ulcers generally involves injection therapy, thermal therapy, and mechanical therapies which currently impose significant limits as these treatments are generally expensive, time intensive and limited in the surface area of tissue that can be treated. The only solution generally available for chronic ulcer management is gastric bypass which has various limitations, costs and drawbacks of its own.

### SUMMARY

The present disclosure relates to a device for treating tissue. The device includes a cap, a patch deployment mechanism, and a first therapeutic patch. The cap is configured to be coupled to a distal end of a flexible insertion device. The cap includes a wall defining a chamber with an open distal end. The patch deployment mechanism is slidably received within the chamber in an insertion configuration. The patch deployment mechanism includes a proximal member coupled to a distal member by a plurality of bowing members. The bowing members is configured to expand radially outward relative to the insertion configuration when longitudinally compressed via movement of the distal member toward the proximal member.

The first therapeutic patch is wrapped around the patch deployment mechanism. The first therapeutic patch is configured to be forced radially outward into contact with and adhere to a first target portion of tissue when the patch deployment mechanism is longitudinally compressed and the bowing members are radially expanded. The patch deployment mechanism is configured to radially retracted after the first therapeutic patch has been adhered to the first target portion of tissue, from a deployed configuration to the insertion configuration to separate the first therapeutic patch from the patch deployment mechanism leaving the first therapeutic patch in a desired position adhered to the first target portion of tissue.

In an embodiment, the device further includes a control wire coupled to the distal member of the patch deployment mechanism, movement of the control wire proximally relative to the patch deployment mechanism compressing the patch deployment mechanism along a longitudinal axis thereof and expanding the bowing members radially away from the longitudinal axis.

In an embodiment, the device further includes a tube slidably receiving the control wire. The tube is coupled to the proximal member of the patch deployment mechanism so that movement of the control wire within the tube moves the distal member of the patch deployment mechanism relative to the distal member of the patch deployment mechanism to move the patch deployment mechanism between the insertion configuration and a radially expanded configuration.

In an embodiment, the device further includes a handle including a slider coupled to the control wire. The slider is configured so that movement of the slider relative the tube moves the control wire through the tube to move the patch deployment mechanism between the insertion configuration and the radially expanded configuration.

In an embodiment, the handle further includes a handle body and a spool slidably mounted on the handle body, the spool being coupled to the tube so that movement of the spool relative to the handle body moves the patch deployment mechanism relative to the cap.

In an embodiment, each of the bowing members is coupled to a distal surface of the proximal member and wherein each of the bowing members is coupled to a proximal surface of the distal member, the bowing members being arranged, in the insertion configuration, in a cylinder extending about a longitudinal axis of the patch deployment mechanism.

In an embodiment, the bowing members are coupled to the proximal and distal members radially within radially outer surfaces of the proximal and distal members with respect to the longitudinal axis of the patch deployment mechanism and wherein a distance between the radially outer surfaces of the bowing members and the radially outer surfaces of the proximal and distal members is selected to provide a clearance between the bowing members and the radially outer surfaces of the proximal and distal members to accommodate the first therapeutic patch.

In an embodiment, the proximal and distal members are substantially circular and are parallel to one another and centered about the longitudinal axis of the patch deployment mechanism to form a cylinder and wherein the cap is cylindrical and sized to slidably receive the patch deployment mechanism therein when the patch deployment mechanism is in the insertion configuration and wherein, in a radially expanded configuration, a maximum diameter defined by the bowing members is greater than an outer diameter of the cap.

In an embodiment, the bowing members are formed of one of Nitinol, Nitional cobalt alloy wire, pebax, nylon, ptfe, stainless steel, hypotube and a biocompatible hard plastic.

In an embodiment, the device further includes a second therapeutic patch wrapped around the patch deployment mechanism and radially outside the first therapeutic patch. The second therapeutic patch is configured to be forced radially outward into contact with and adhere to a second target portion of tissue when the patch deployment mechanism is moved to a radially expanded configuration. The patch deployment mechanism is configured to be moved, after the second therapeutic patch has been adhered to the second target portion of tissue as desired, from the radially expanded configuration to the insertion configuration to separate the second therapeutic patch from the patch deployment mechanism leaving the second therapeutic patch in a desired position adhered to the second target portion of tissue.

In an embodiment, the first therapeutic patch has a width extending along a longitudinal axis of the patch deployment mechanism and a length transverse to the width, the first therapeutic patch being wrapped around the patch deployment mechanism so that the length of the first therapeutic patch extends circumferentially around the patch deployment mechanism, the length of the first therapeutic patch being configured to be at least as great as a diameter of the patch deployment mechanism in the deployed configuration.

In an embodiment, the first therapeutic patch includes an adhesive having a predetermined cure time and wherein the first therapeutic patch is configured to be removable from tissue against which it has been pressed before the predetermined cure time has elapsed since the first therapeutic patch was pressed against the tissue.

In addition, the present disclosure relates to a method for treating tissue. The method includes inserting into a living body a flexible endoscope a device including a cap mounted on a distal end thereof, the device including, slidably received within the cap, a patch deployment mechanism with a first therapeutic patch wrapped therearound, the patch deployment mechanism being maintained during insertion in an insertion configuration in which the patch deployment mechanism and the first therapeutic patch are maintained within the cap; moving the patch deployment mechanism distally out of the cap to expose the patch deployment mechanism and the first therapeutic patch; expanding the patch deployment mechanism to move the first therapeutic patch radially outward into contact with a first target portion of tissue to be treated; and radially contracting the patch deployment mechanism out of contact with the first therapeutic patch to leave the first therapeutic patch in a desired position adhered to the first target portion of tissue.

In an embodiment, the first therapeutic patch has a width extending along a longitudinal axis of the patch deployment mechanism and a length transverse to the width, the first therapeutic patch being wrapped around the patch deployment mechanism so that the length of the first therapeutic patch extends circumferentially around the patch deployment mechanism, the length of the first therapeutic patch being configured to be at least as great as a diameter of the patch deployment mechanism in a deployed configuration. The method further includes the step of pressing the first therapeutic patch against the first target portion of tissue and moving the patch deployment mechanism relative to the first target portion of tissue in a direction corresponding to the length of the first therapeutic patch to unroll the first therapeutic patch from the patch deployment mechanism over the first target portion of tissue until the entire length of the first therapeutic patch has been adhered to the first target portion of tissue.

In an embodiment, the device further includes a second a therapeutic patch wrapped around the first therapeutic patch and the patch deployment mechanism.

In an embodiment, the patch deployment mechanism includes a plurality of bowing members which, in the insertion configuration form a cylinder having a diameter less than an inner diameter of the cap, further comprising, longitudinally compressing the patch deployment mechanism to radially expand the bowing members to push the first therapeutic patch radially outward.

In an embodiment, the device includes a distal member of the patch deployment mechanism coupled to a distal end of a control wire that extends to a proximal end that remains outside the living body and a tube coupled to a proximal member of the patch deployment mechanism, further comprising moving the control wire proximally relative to the tube to move the distal member proximally toward to the proximal member to longitudinally compress and radially expand the bowing members.

### BRIEF DESCRIPTION OF DRAWINGS

FIG. 1 shows a perspective view of a distal portion of an assembly for placing a patch on target tissue according to a first illustrative embodiment.

FIG. 2 shows a perspective view of the distal portion of the assembly of FIG. 1 showing a control wire within a coil of the embodiment.

FIG. 3 shows a perspective view of a handle of the first illustrative embodiment.

FIG. 4A shows the handle of FIG. 3 with a spool of the handle moved distally to move a patch deploying mechanism out of a protective cap.

FIG. 4B shows the handle of FIG. 4A with a knob of the handle moved proximally relative to the spool to longitudinally compress and radially expand the patch deploying mechanism.

FIG. 5 shows the patch deploying mechanism of the assembly of FIG. 1 longitudinally compressed and radially expanded.

FIG. 6 shows a perspective view of a distal portion of an assembly for placing a patch on target tissue according to a second illustrative embodiment.

FIG. 7 shows a perspective view of a handle of the second illustrative embodiment.

FIG. 8 shows a perspective view of the distal portion of the assembly of FIG. 6 showing a control wire within a coil of the embodiment.

FIG. 9 shows a patch deploying mechanism of the assembly of FIG. 6 longitudinally compressed and radially expanded.

FIG. 10 shows the patch deploying mechanism of FIG. 9 deploying a patch.

### DETAILED DESCRIPTION

The embodiments described herein relate to apparatus and methods for the minimally invasive application of a patch of material to tissue within a living body. Although specific embodiments will be described in regard to the endoscopic application of a patch or patches of material to treat tissue within the digestive tract (e.g., esophagus, stomach, small intestine and colon, common bile duct and pancreatic ducts), those skilled in the art will understand that the system apparatus and techniques may be employed to apply a patch or patches of material to tissue within any other internal bodily tissues. Furthermore, as used in this application the terms distal and proximal refer to directions along a device from parts closer to a user (proximal) and further (distal) from the user. In general, the proximal end of such a device will include a handle which, during use of the device, remains outside the body accessible to the user and an end effector at the distal end which is inserted into the body (e.g., via a naturally occurring bodily orifice and navigated to a target site adjacent to target tissue to be treated via a natural body lumen).

However, those skilled in the art will understand that other tissue that may be accessed within the body via other pathways (e.g., along a path that proceeds through a natural body lumen to a point at which the device is passed out of the lumen (e.g., via a surgical opening formed therein) and that this tissue may then be treated by the application of a patch in the same manner described for the application of a patch to tissue on the inner wall of the lumen. Furthermore, embodiments will be described in which a therapeutic patch is applied to target tissue to achieve or assist in achieving hemostasis. However, as those skilled in the art will understand, such a therapeutic patch may be applied using the same apparatus and techniques where the patch is configured to treat conditions different from or the bleeding/tissue opening sealing function of the described embodiments.

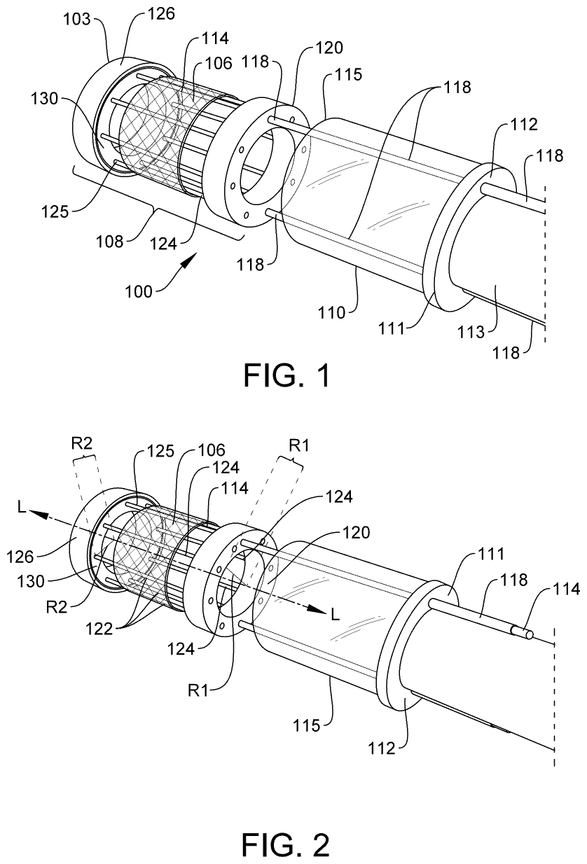

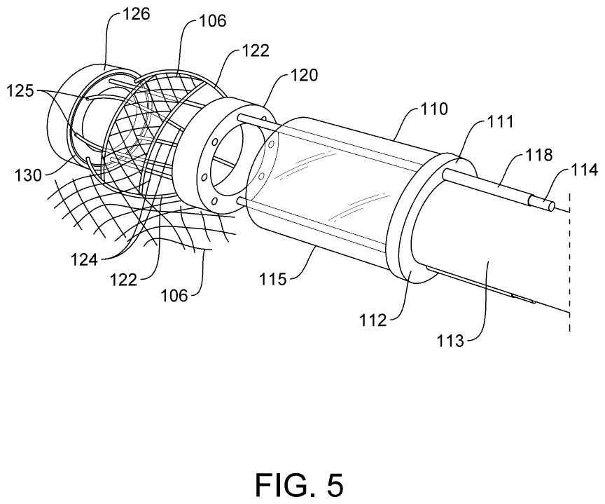

FIGS. 1-5 show a device 100 extending from a proximal portion 102 including a handle 104 to a distal portion 103 including a therapeutic patch 106 that is wrapped around an expanding element (e.g., a deployment mechanism 108). A cap 110 extends distally from a proximal end 112 configured to be coupled to a distal end of an insertion device (e.g., an endoscope 113) to a distal end with a length of the cap 110 being selected to be sufficient to accommodate the deployment mechanism 108 therein to protect the patch 106 (e.g., from exposure to bodily fluids) during insertion of the device 100 to a target location in the body. That is, in an insertion configuration (i.e., a first position) the deployment mechanism 108 will be received within the cap 110 so that the cap 110 covers the patch 106. Those skilled in the art will understand that, due to size constraints and to limit the entry of bodily fluids into the cap 110 before the patch 106 has been extended distally out of the cap 110, the outer diameter of the cylinder formed by the patch 106 wrapped around the deployment mechanism 108 is, in an exemplary embodiment selected to closely match the inner diameter of the cap 110.

As can be seen in FIG. 2, the proximal end of the cap 110 of this embodiment is formed as a ring 111 that is sized and shaped to be slidably received and retained in a desired position on the end of the endoscope 113, e.g., via a friction fit. A transparent cylindrical sheath 115 that extends distally from the ring 111 is sized and shaped to accommodate the deployment mechanism 108 therein in the insertion configuration. The distal end of the sheath 115 is open so that the deployment mechanism 108 may be advanced distally out of the cap 110 when the device has been inserted to a target site within the body (e.g., adjacent to a portion of tissue to be treated by the device 100).

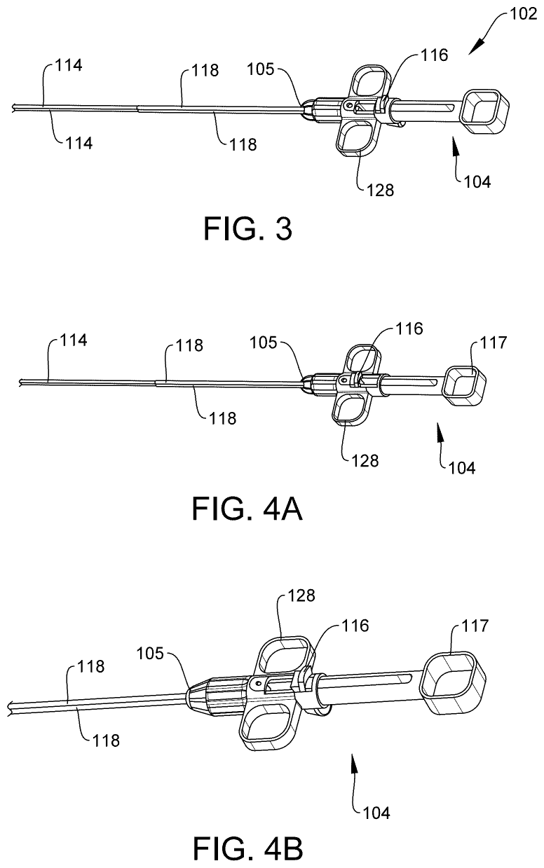

As seen in FIGS. 3, 4A and 4B, the device 100 includes a pair of control wires 114 each of which extends from a proximal end coupled to a slider 116 of the handle 104 with each of the control wires 114 extending distally into a corresponding tube 118. Each of the tubes 118 is coupled to a spool 128 that is slidably attached to a body of the handle 104 for movement relative to a thumb ring 117 of the handle 104. The slider 116 is slidably mounted on the spool 128 so that the slider 116 may move relative to the thumb ring 117 along with the spool 128 and may move relative to the spool 128 and the thumb ring 117. Each control wire 114 is slidably received within its corresponding tube 118 and distal ends 119 of the tubes 118 are coupled to a proximal ring 120 at the proximal end 112 of the deployment mechanism 108. In this embodiment, the distal ends 119 of the tubes 118 are coupled to diametrically opposed points on the proximal ring 120.

However, those skilled in the art will understand that the distal ends 119 of the tubes 118 may be coupled to the proximal ring 120 at any locations so long as the functioning of the tubes 118 (e.g., moving the deployment mechanism 108 in and out of the cap 110) and the functioning of the control wires 114 (described below) is not impeded. The tubes 118 pass through a distal end 105 of the handle 104 to couple to the spool 128. Thus, movement of the spool 128 (proximally or distally) relative to the rest of the handle 104 moves the tubes 118 either proximally or distally. In use, the cap 110 with the deployment mechanism 108 received therein is mounted to the distal end of the endoscope 113 with the tubes 118 extending proximally along the endoscope 113 to the handle 104.

Thus, as the spool 128 is moved distally relative to the thumb ring 117 of the handle 104, the tubes 118 are extended distally relative to the handle 104 (so that the distal ends 119 of the tubes 118 are moved further away from the handle 104) and relative to the endoscope 113 and the cap 110. Thus, when the spool 128 is moved distally away from the thumb ring 117, the deployment mechanism 108 is extended distally out of the cap 110 while proximal movement of the spool 128 would retract the deployment mechanism 108 proximally back into the cap 110.

Each of the control wires 114 passes through a corresponding opening the proximal ring 120 and extends distally to couple to a distal ring 126 of the deployment mechanism 108. In this embodiment, the control wires 114 are coupled to diametrically opposed points on a proximal face of the distal ring 126. However, those skilled in the art will understand that the distal ends of the control wires 114 may be coupled to the distal ring 126 at any locations so long as the functioning of the control wires 114 (described below) is not impeded. As will be described in more detail below, if the position of the slider 116 is maintained constant relative to the spool 128, the distance between the proximal ring 120 and the distal ring 126 of the deployment mechanism 108 will remain constant. If the spool 128 is moved relative to the thumb ring 117, the control wires 114 will be moved correspondingly within the tubes 118 while contact between the tubes 118 and the proximal ring 120 maintains the position of the proximal ring 120 constant. Thus, as will be described below, drawing the slider 116 proximally relative to the spool 128 will pull the distal ring 126 proximally toward the proximal ring 120.

The deployment mechanism 108 includes a plurality of bowing members 122 each of which extends between a proximal end 124 coupled to the distal side of the proximal ring 120 and a distal end 125 coupled to a proximal side of the distal ring 126. The bowing members 122 are configured so that, in a pre-deployment position, the bowing members 122 extend along substantially straight lines from the proximal ring 120 to the distal ring 126. The proximal and distal rings 120, 126 of the deployment mechanism 108 of this embodiment are cylindrical, parallel to one another and of the same diameter with the centers of the proximal ring 120 and the distal ring 126 being located on the same axis L1. Thus, the proximal ring 120 and the distal ring 126 of this embodiment define a cylinder having an outer diameter equal to the outer diameter of the proximal and distal rings 120, 126.

The proximal ends 124 of the bowing members 122 of this embodiment are coupled to the distal side of the proximal ring 120 along a first circle (i.e., a R₁ distance between the axis L and a point at which each of the proximal ends 124 of the bowing members 122 the proximal ring 120 is the same for each of the bowing members 122). The distal ends 125 of the bowing members 122 of this embodiment are coupled to the proximal side of the distal ring 126 along a second circle (i.e., a distance R₂ between the axis L and a point at which each of the distal ends of the bowing members 122 the distal ring 126 is the same for each of the bowing members 122). The distances R₁ and R₂ are, in this embodiment, selected to be equal to one another and smaller than the outer diameters of the proximal ring 120 and the distal ring 126 by a selected clearance amount so that, when a patch 106 is wound about the bowing members 122, the outer diameter of the patch 106 (in the pre-deployment position) is no greater than the outer diameter of the cylinder defined by the proximal ring 120 and the distal ring 126.

However, those skilled in the art will understand that any other arrangement of the bowing members 122 may be employed so long as the patch 106 can be accommodated within the cap 110. As indicated above, the tubes 118 are fixed to the proximal ring 120 while the control wires 114 pass through the proximal ring 120 to couple to the distal ring 126. The bowing members 122 are formed of a flexible material (e.g., Nitinol) that is configured to bow radially outward when the distance between the proximal ring 120 and the distal ring 126 is compressed. Thus, when the deployment mechanism 108 is extended distally out of the cap 110 and the slider 116 is moved proximally relative to the spool 128, the distal ring 126 is drawn toward the proximal ring 120 and the bowing members 122 are compressed longitudinally and bowed radially outward. This, in turn, pushes the patch 106 radially outward to a diameter greater than a diameter of the proximal ring 120 and the distal ring 126.

To place the device 100 in the insertion configuration, the deployment mechanism 108 is drawn proximally into the cap 110 until the entirety of the deployment mechanism 108 and the patch 106 (or alternatively, at least the distal end of the patch 106) is received within the cap 110. When the endoscope 113 (or other insertion device) has been maneuvered so that the distal end of the cap 110 is positioned relative to the target tissue as desired, the user pushes the spool 128 distally so that the deployment mechanism 108 is extended distally out of the distal end of the cap 110.

As would be understood by those skilled in the art, when the deployment mechanism 108 is moved out of the constraint of the cap 110, the bowing members 122 may, in certain embodiments, begin to expand radially outward even before the user has operated the slider 116 (e.g., through any number of mechanisms such as a bias imparted to the members forming the stent, a memorized shape imparted to a shape memory material such as Nitinol, etc.). This will begin to expand the patch 106 radially outward toward the target tissue. As would be understood by those skilled in the art, this self-expansion of the deployment mechanism 108 may, in certain circumstances, be sufficient to move an outer surface of the patch 106 radially outward into contact with the target tissue.

When the target tissue forms part of the wall of a tubular organ (e.g., esophagus, colon, small intestine), the outward pressure applied by the deployment mechanism 108 urging the patch 106 into contact with the target tissue may be sufficient to adhere the patch 106 to the target tissue. The requirements for achieving a desired adherence of the patch 106 to target tissue and the characteristics of the patch 106 and the expansion apparatus employed to achieve this adherence will be described in more detail below.

As indicated above, the proximal ring 120 is coupled to the distal ends of the tubes 118. Thus, after the deployment mechanism 108 and the patch 106 have been extended distally out of the cap 110, the user may draw the slider 116 proximally relative to the spool 128 of the handle 104 to pull the distal end of the control wire 114 proximally relative to the cap 110. As indicated above, this will draw the distal ring 126 proximally toward the proximal ring 120 while the proximal ring 120 is maintained in its position (i.e., as the spool 128 remains in a constant position relative to the thumb ring 117). This compresses the bowing members 122 longitudinally (i.e., the distance between the distal and proximal ends 125, 124 of each of the bowing members 122 is reduced along the axis L).

This causes the deployment mechanism 108 to expand radially outward driving the patch 106 wrapped around the deployment mechanism 108 to expand radially as well. As seen in FIG. 1 and FIG. 2, a proximal surface of the distal ring 126 includes a slot 130 extending distally into a radially outer portion of a proximal side of the distal ring 126 so that the patch 106 can be inserted therein and the patch 106 will lie flush with the radially outer surface of the distal ring 126 when the patch 106 is wrapped around the deployment mechanism 108. As would be understood by those skilled in the art, a similarly sized, shaped and positioned slot may be formed at a radially outer edge of the distal end of the proximal ring 120 so that the patch 106, when wrapped about the deployment mechanism 108 does not increase the outer diameter of the deployment mechanism beyond the limit already established by the radially outer-most surfaces of the proximal and distal rings 120, 126, respectively.

Those skilled in the art will understand that the patch 106 of this embodiment is wrapped around the deployment mechanism 108 loosely so that it can freely unwind as the deployment mechanism 108 expands—i.e., so that the patch 106 loosens instead of ripping or impeding expansion of the deployment mechanism 108. In this embodiment, the patch 106 is wrapped around the deployment mechanism 108 for more than a single circumference of the deployment mechanism 108 so that as the patch 106 is expanded from its initial state in the insertion configuration the patch 106 continues to cover the entire circumference of the expanded deployment mechanism 108. That is the patch 106 is wrapped more than 360 degrees around the deployment mechanism 108 to permit for the radial expansion of the patch 106 which permits the material of the patch 106 to maintain full circumferential coverage of the deployment mechanism 108 as the radius of the deployment mechanism 108 (and the circumference of the deployment mechanism 108) expands.

As would be understood by those skilled in the art, this type of mechanical longitudinal compression of the deployment mechanism 108 can apply significant radially outward pressure as the longitudinal force exertable by a user via the slider 116 is considerable. For example, using the slider 116, a user may generate an outward radial force of from 1 to 15 lbf which, as those skilled in the art will understand, corresponds, due to known mechanical advantage, to a radially outward force of 5-20 lbf applied by expansion of the deployment mechanism 108 to the patch 106 and the surrounding tissue.

In an exemplary embodiment, the cap 110 may have an outer diameter ranging from approximately 3 mm to approximately 20 mm and an inner diameter ranging from approximately 2.5 mm to approximately 17.5 mm. The proximal end 112 of the cap 110 may have an outer diameter of approximately 18 mm and a thickness of approximately 2.5 mm. The proximal ring 120 may have an outer diameter of approximately 17 mm, an inner diameter of approximately 11 mm and a thickness of approximately 2.5 mm. The distal ring 120 may have an outer diameter of approximately 17 mm, an inner diameter of approximately 11 mm and a thickness of approximately 2.5 mm. The patch 106 may have a length ranging from approximately 1 inch to approximately 2 inches, a width ranging from approximately 1 inch to approximately 2 inches and a thickness ranging from approximately 0.2 mm to approximately 0.5 mm. Each of the bowing members 122 may have a diameter ranging from approximately 0.1 mm to approximately 0.2 mm and a length of approximately 28 mm.

In the non-expanded configuration (i.e., insertion configuration), the deployment mechanism 108 may have a diameter ranging from approximately 2.5 mm to approximately 15 mm. In the expanded configuration (i.e., deployment configuration), the deployment mechanism 108 may have a diameter ranging from approximately 15 mm to approximately 32 mm. As one with ordinary skill in the art will ascertain, the above dimensions are exemplary and may be modified in further embodiments.

A technique for applying a patch 106 to larger circumferential portions of tissue or to partially circumferential portions of tissue in large diameter spaces (spaces having a diameter beyond the maximum radial expansion of the patch 106 achievable by manipulation of the deployment mechanism 108) will be described in more detail below. Those skilled in the art will understand that all of the dimensions given here are illustrative and if, for example, an insertion device presents other dimensions and/or anatomical considerations present challenges other dimensions may be employed as necessary.

As would be understood by those skilled in the art the patch 106 may be a biocompatible, flexible and resorbable gelatin patch configured to begin a coagulation cascade via mechanical interaction. For example, in one embodiment, the patch 106 is formed of one or more Chitosan sheaths. As would be understood by those skilled in the art, Chitin and its deacetylated derivative, chitosan, are a family of linear polysaccharides including varying amounts of (β1→4) linked residues of N-acetyl-2 amino-2-deoxy-D-glucose (glucosamine, GlcN) and 2-amino-2-deoxy-D-glucose (N-acetyl-glucosamine, GlcNAc) residues. The size of the patch 106 may vary depending on the application and the target anatomy. As an example, the patch 106 may have a size of, for example, 10″×10″ but may have any size up to 20″×20″. The patch 106 may be used, for example, to stop the bleeding/cover bleeding ulcers even where larger surface areas of target tissue need to be treated.

When the patch 106 is formed of Chitosan, the patch 106 may be repositioned. That is, if after the patch 106 has been initially placed on target tissue, the user believes the position is not correct (e.g., based on observation via the endoscopic vision system), the user may remove the patch 106 from the target tissue before adhesive in the patch 106 has cured. That is, after the patch 106 is pushed against the tissue, adhesive in the patch 106 takes a certain amount of time to adhere to the tissue (i.e., a known curing time must elapse when the patch 106 is in contact with body fluid and tissue before the patch 106 is adhered thereto).

Thus, if the user sees that the patch 106 (e.g., under direct visualization via the scope vision system) has been placed in contact with tissue in a position that is not correct, the user may can retract the deployment mechanism 108 radially inward (by pushing the spool 128 distally relative to the thumb ring 117) to extend the control wire 114 distally pushing the distal ring 126 distally away from the proximal ring 120 so that the bowing members 122 are drawn radially inward pulling the patch 106 radially inward out of contact with the tissue. As the deployment mechanism 108 retracts radially inward, the patch 106 rolls back on the deployment mechanism 108 due to its stiffness. The user then repositions the deployment mechanism 108 and the patch 106 by moving the cap 110 and/or by altering the aim of the distal end of the endoscope until the proper position of the patch 106 relative tissue has been achieved. The user may then reapply the patch 106 in the same manner described above.

In a similar manner, a user may use the same deployment mechanism 108 to place the multiple patches 106 over different target portions of tissue at different locations. For example, instead of a single patch 106 rolled around the deployment mechanism 108 as a single continuous sheet, any number of patches can be rolled around the deployment mechanism 108 and separately placed on different portions of tissue. In such an embodiment, a first patch 106 is rolled around the deployment mechanism 108 from a first end to a free end and a second patch (either not connected to the first patch or releasably coupled thereto) is wrapped around the deployment mechanism 108 in the same direction as the first patch starting from a first end adjacent to the free end of the first patch so that the second patch covers the first patch.

Subsequent patches may then be wrapped further around the deployment mechanism 108 with each subsequent patch 106 starting adjacent to the exposed end of the previous patch 106 and laying over the top of this immediately previous patch 106 and so on. To place these individual patches 106, a user positions the deployment mechanism 108 and the patches 106 as desired relative to a first target portion of tissue to be treated. The user expands the deployment mechanism 108 as described above to expand an outermost one of the patches into contact with this first target portion of tissue and, when this patch has properly adhered to the first target portion of tissue as desired, the user extends the control wire 114 distally to radially retract the deployment mechanism 108 until the second patch immediately radially within the outermost patch is moved out of contact with the outermost patch. The user then positions the second patch in a desired location relative to a second portion of tissue to be treated and adheres the second patch to the second portion of tissue in the same manner described above for the outermost patch. The process is repeated in the same manner for subsequent patches or until the desired number of patches have been placed as desired.

Alternatively, the patch 106 may be an Electrospun patch including nano fibers or formed of a biocompatible material such as nylon, urethane, nylon or latex) capable of forming a flexible collapsible member, or membrane as would be understood by those skilled in the art. The patch 106 may also include any combination of these materials as well as any other bioadhesive materials such as chitosan, modified chitosan, cellulose, pHEMA, PVA, PEG, or composites of these polymers. The patch may alternatively be formed as a mesh of Polypropylene, Polyester and/or ePTFE. In addition, fibrin glue or any other suitable adherent may be employed on the outer layer of patch. As the fibrin glue comes in contact with the tissue, it helps to attach the patch to the tissue.

In an exemplary embodiment, the bowing members 122 may be formed of stainless steel or from portions of a hypotube. Alternatively, the flexible portions may be formed of any one or a combination of Nitional cobalt alloy wire, plastics made of pebax, nylon, or ptfe, etc. and the stiff portion may alternatively be formed of any of a number of biocompatible hard plastics such as ABS.

If the target tissue is a portion of a body lumen of less than a predetermined maximum diameter (e.g., the diameter to which the deployment mechanism may radially expand the patch 106 to press against the target tissue with a desired force) (e.g., a force sufficient to ensure a desired adherence between the patch 106 and the target tissue), the user may then expand the deployment mechanism 108 to force the patch 106 radially outward into contact with a fully circumferential portion of the tissue extending proximally and distally beyond the ends of the target portion of tissue so that the entire portion of target tissue will be covered by the patch 106.

If the portion of tissue to be treated is not in a vessel having a lumen of a size admitting of full circumferential application of the patch 106 via expansion of the deployment mechanism 108 alone (e.g., if the target portion of tissue is a part of a wall of a larger vessel such as the stomach), the user, after expansion of the deployment mechanism, will manipulate the endoscope to press a first portion of the patch 106 against the wall of the tissue. The user would locate a free end of the patch 106 (e.g., by visually identifying a marker formed on the patch 106 at the free edge and place this free edge in contact with the tissue and unroll the patch 106 in a direction opposite the direction in which the patch 106 was rolled onto the deployment mechanism (e.g., deployment mechanism 108).

The user may also articulate and manipulate the endoscope to apply radial pressure urging the patch 106 into contact with the tissue and may even articulate the endoscope to add to the force applied to the patch 106 as the deployment mechanism 108 is slowly expanded. The user would then move the deployment mechanism 108 so that the patch 106 and the deployment mechanism 108 are rolled over the surface of the target portion of tissue to be treated. That is, the user would move the deployment mechanism 108 in a direction perpendicular to the longitudinal axis L while maintaining contact with the tissue so that, as the patch 106 adheres to the tissue, the patch 106 is unspooled from the deployment mechanism 108 in a direction opposite a direction in which the patch 106 was rolled onto the deployment mechanism.

In this manner, the user may unspool a length of patch 106 that is greater than a diameter of the expanded deployment mechanism 108 to treat larger portions of target tissue. That is, if the patch 106 is wrapped around the deployment mechanism 3 times, the user may unspool this entire length of patch 106 to treat a portion of tissue much larger than the surface area of the expanded deployment mechanism 108. The deployment mechanism 108 applies line contact forcing the patch 106 radially outward against the target tissue. As the deployment mechanism 108 expands, the radial pressure on the patch 106 perpendicular to the longitudinal axis of the deployment mechanism 108 unwraps the patch 106. In such a use, the deployment mechanism 108 may be held stationary while the user manipulates the endoscope to unwrap the patch 106 from the deployment mechanism 108 as each portion of the patch 106 becomes attached to tissue.

It will also be understood by those skilled in the art that the patch 106 (or plurality of patches 106) is formed to a desired length that is related to a length of the deployment mechanism 108 when it is longitudinally extended so that, as the deployment mechanism 108 is longitudinally compressed and radially expanded, the length of the longitudinally compressed deployment mechanism 108 is sufficient to press a target portion of the patch 106 against the target tissue as desired. That is, depending on several factors including, for example, a stiffness of the patch 106, portions of the patch 106 extending longitudinally beyond a length of the deployment mechanism 108 (in its radially extended state) will radially expand even though they are not being pressed outward from within. That is, for patches 106 of a certain stiffness, portions of the patch 106 not resting on the outer surface of the radially expanded deployment mechanism 108 will expand outward when those portions of the patch 106 that are in contact with the deployment mechanism 108 are pressed outward as the stiffness of the patch 106 allows these extended portions to cantilever outward from the supported portions of the patch 106 to press against the target tissue.

As would be understood by those skilled in the art, the length at which these cantilevered portions of the patch 106 will be properly adhered to the target tissue will depend on the stiffness of the patch 106, the properties of the adhesive material in the patch 106, properties of the target tissue, etc. If desired, after a first portion of a patch has been adhered to a first portion of tissue, the user may longitudinally extend and radially retract the deployment mechanism 108 and move the radially contracted deployment mechanism 108 to a new position inside the expanded patch 106. The user may then re-expand the deployment mechanism 108 to press a new portion of the patch 106 into contact with a second portion of tissue. The user may repeat this procedure as often as needed to achieve the desired adherence between the entire patch 106 and the underlying tissue.

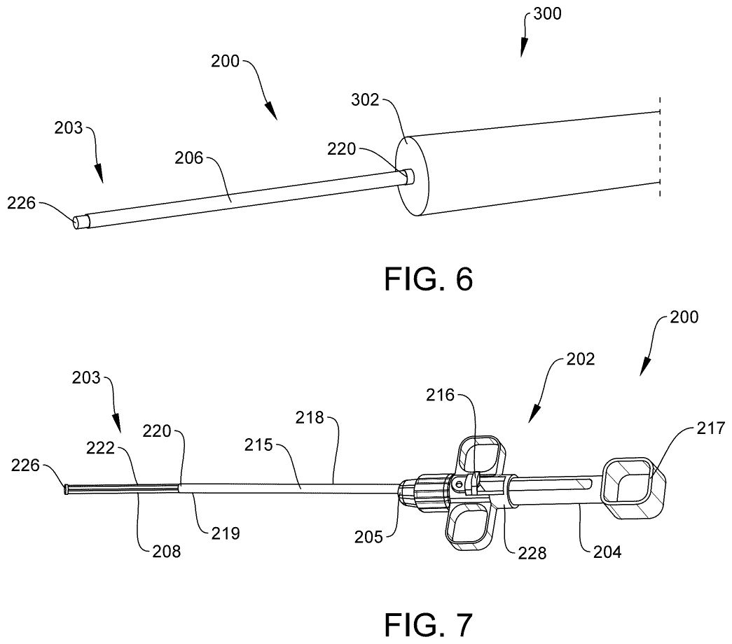

FIGS. 6-10 show a further exemplary embodiment of the present disclosure including a device 200. The device 200 extends from a proximal portion 202 including a handle 204 to a distal portion 203 including a therapeutic patch 206 that is wrapped around an expanding element (e.g., a deployment mechanism 208). In the exemplary embodiment, the patch 206 is substantially similar to the patch 106 above and the deployment mechanism 208 is substantially similar to the deployment mechanism 208. For example, the requirements for achieving a desired adherence of the patch 206 to target tissue at a target site and the characteristics of the patch 206 are substantially similar to the requirements for achieving the desired adherence of the patch 106 and the characteristics of the patch 106.

The deployment mechanism 208 extends from a proximal end 212 to a distal end 213 and is configured to be inserted through a working channel of an insertion device (e.g., an endoscope 300) until a distal end 302 of the endoscope 300 reaches a target site within the body (e.g., adjacent to a portion of tissue to be treated by the device 200). That is, in an insertion configuration (i.e., a first position) the deployment mechanism 208 will be received within the distal end 302 of the endoscope 300 such that the distal end 302 covers the patch 206 (e.g., to protect the patch 206 from exposure to bodily fluid). Those skilled in the art will understand that, due to size constraints and to limit the entry of bodily fluids into the working channel before the patch 206 has been extended distally out of the working channel, the outer diameter of the cylinder formed by the patch 206 wrapped around the deployment mechanism 208 may be selected to closely match the inner diameter of the working channel.

In a further exemplary embodiment, the device 200 may include an elongated coil 215 that extends distally from the proximal end 202. The coil 215 is sized and shaped to accommodate the deployment mechanism 208 therein in the insertion configuration. A distal end of the coil 215 is open so that the deployment mechanism 208 may be advanced distally out of the coil 215 when the device 200 has been inserted to the target site.

In a further exemplary embodiment, instead of the coil 215 and similar to device 100, the device 200 may include a cap extending distally from a proximal end configured to be coupled to the distal end 302 of the endoscope 300. A length of the cap is selected to be sufficient to accommodate the deployment mechanism 208 therein to protect the patch 206. The cap may include a transparent cylindrical sheath configured to accommodate the deployment mechanism 208 therein, a distal end of the sheath being open so that the deployment mechanism 208 may be advanced distally out of the cap. In the further embodiment, the outer diameter of the cylinder formed by the patch 206 wrapped around the deployment mechanism 208 may be selected to closely match the inner diameter of the cap.

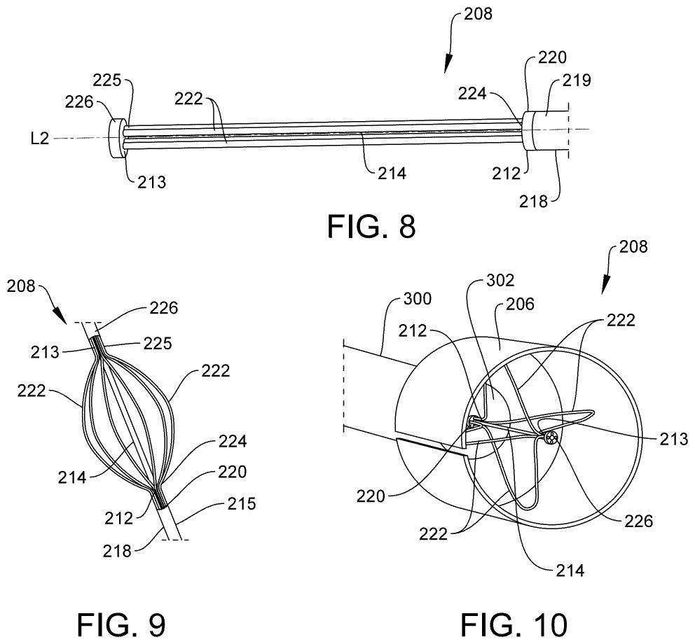

As seen in FIGS. 8-10, the device 200 includes a control wire 214 extending from a proximal end coupled to a slider 216 of the handle 204. The control wire 214 extends through a tube 218 coupled to a spool 228 that is slidably attached to a body of the handle 204 for movement relative to a thumb ring 217 of the handle 204. The slider 216 is slidably mounted on the spool 228 so that the slider 216 may move relative to the thumb ring 217 along with the spool 228 and may move relative to the spool 228 and the thumb ring 217. The control wire 214 is slidably received within the tube 218, a distal end 219 of the tube 218 being coupled to a proximal ring 220 at a proximal end 212 of the deployment mechanism 208.

The tube 218 passes through a distal end 205 of the handle 204 to couple to the spool 228. Thus, movement of the spool 228 (proximally or distally) relative to the rest of the handle 204 moves the tube 218 either proximally or distally. Thus, as the spool 228 is moved distally relative to the thumb ring 217 of the handle 204, the tube 218 is extended distally relative to the handle 204 (so that the distal end 219 of the tube 218 is moved further away from the handle 204) and relative to the endoscope 300 and the coil 215. Thus, when the spool 228 is moved distally away from the thumb ring 217, the deployment mechanism 208 is extended distally out of the coil 215 while proximal movement of the spool 228 would retract the deployment mechanism 208 proximally back into the coil 215.

The control wire 214 passes through a corresponding opening in the proximal ring 220 and extends distally to couple to a distal ring 226 at the distal end 213 of the deployment mechanism 208. In this embodiment, the corresponding opening in the proximal ring 220 extends through a center of the proximal ring 220 and the control wire 214 is couples to a center point on a proximal face of the distal ring 226. However, those skilled in the art will understand that the distal end of the control wire 214 may be coupled to the distal ring 226 at any location so long as the function of the control wire 214 (described below) is not impeded. As will be described in more detail below, if the position of the slider 216 is maintained constant relative to the spool 228, the distance between the proximal ring 220 and the distal ring 226 of the deployment mechanism 208 will remain constant. If the spool 228 is moved relative to the thumb ring 217, the control wire 214 will be moved correspondingly within the tube 218 while contact between the tube 218 and the proximal ring 220 maintains the position of the proximal ring 220 constant. Thus, as will be described below, drawing the slider 216 proximally relative to the spool 228 will pull the distal ring 226 proximally toward the proximal ring 220.

The deployment mechanism 208 includes a plurality of bowing members 222 each of which extends between a proximal end 224 coupled to a distal side of the proximal ring 220 and a distal end 225 coupled to a proximal side of the distal ring 226. The bowing members 222 are configured so that, in a pre-deployment position, the bowing members 222 extend along substantially straight lines from the proximal ring 220 to the distal ring 226. The proximal and distal rings 220, 226 of the deployment mechanism 208 of this embodiment are cylindrical, parallel to one another and of the same diameter with the centers of the proximal ring 220 and the distal ring 226 being located on the same axis L2. Thus, the proximal ring 220 and the distal ring 226 of this embodiment define a cylinder having an outer diameter equal to the outer diameter of the proximal and distal rings 220, 226. Although the exemplary embodiment in FIG. shows the deployment mechanism 208 having four bowing members 222, further embodiments may have any number of bowing members 222 as long as the bowing members 222 are able to contract longitudinally and expand radially.

The proximal ends 224 of the bowing members 222 of this embodiment are coupled to the distal side of the proximal ring 220 along a first circle in a substantially similar manner as the proximal ends 124 of the bowing members 122 are coupled to the distal side of the proximal ring 120. The distal ends 225 of the bowing members 222 of this embodiment are coupled to the proximal side of the distal ring 226 along a second circle in a substantially similar manner as the distal ends 125 of the bowing members 122 are coupled to the proximal side of the distal ring 126. The deployment mechanism 208 is configured such that, when the patch 206 is wound about the bowing members 222, the outer diameter of the patch 206 (in the pre-deployment position) is no greater than the outer diameter of the cylinder defined by the proximal ring 220 and the distal ring 226.

However, those skilled in the art will understand that any other arrangement of the bowing members 222 may be employed so long as the patch 206 can be accommodated within the coil 215. As indicated above, the tube 218 is fixed to the proximal ring 220 while the control wire 214 passes through the proximal ring 220 to couple to the distal ring 226. The bowing members 222 are formed of a flexible material (e.g., Nitinol) that is configured to bow radially outward when the distance between the proximal ring 220 and the distal ring 226 is compressed, as shown in FIG. 9. Thus, as shown in FIG. 10, when the deployment mechanism 208 is extended distally out of the coil 215 and the slider 216 is moved proximally relative to the spool 228, the distal ring 226 is drawn toward the proximal ring 220 and the bowing members 222 are compressed longitudinally and bowed radially outward. This, in turn, pushes the patch 206 radially outward to a diameter greater than a diameter of the proximal ring 220 and the distal ring 226, such that the patch 206 may be applied at the target site.

To place the device 200 in the insertion configuration, the deployment mechanism 208 is drawn proximally into the endoscope 300 until the entirety of the deployment mechanism 208 and the patch 206 (or alternatively, at least the distal end of the patch 206) is received within the endoscope 300. When the endoscope 300 (or other insertion device) has been maneuvered so that the distal end 302 of the endoscope 300 is positioned relative to the target site as desired, the user pushes the spool 228 of the handle 204 distally so that the deployment mechanism 208 is extended distally out of the distal end 302 of the endoscope 300.

Since the proximal ring 220 is coupled to the distal end of the tube 218, after the deployment mechanism 208 and the patch 206 have been extended distally out of the endoscope 300, the user may draw the slider 216 proximally relative to the spool 228 to pull the distal end of the control wire 214 proximally relative to the distal end 302 of the endoscope 300. As indicated above, this will draw the distal ring 226 proximally toward the proximal ring 220 while the proximal ring 220 is maintained in its position (i.e., as the spool 228 remains in a constant position relative to the thumb ring 217). This compresses the bowing members 222 longitudinally (i.e., the distance between the distal and proximal ends 225, 224 of each of the bowing members 222 is reduced along the axis L2). This causes the deployment mechanism 208 to expand radially outward driving the patch 206 wrapped around the deployment mechanism 208 to expand radially as well, as shown in FIG. 10.

Those skilled in the art will understand that various modifications may be made to the disclosed embodiments without departing from the scope of this disclosure which is intended to be limited only by the scope of the claims appended hereto. For example, any of the various components of the several embodiments may be combined in any manner not specifically disclaimed in this disclosure.

1-12. (canceled)

13. A device for treating tissue, comprising:
a cap configured to be coupled to a distal end of a flexible insertion device, the cap including a wall defining a chamber with an open distal end;
a patch deployment mechanism slidably received within the chamber in an insertion configuration, the patch deployment mechanism including a proximal member coupled to a distal member by a plurality of bowing members, the bowing members being configured to expand radially outward relative to the insertion configuration when longitudinally compressed via movement of the distal member toward the proximal member; and
a first therapeutic patch wrapped around the patch deployment mechanism, the first therapeutic patch being configured to be forced radially outward into contact with and adhere to a first target portion of tissue when the patch deployment mechanism is longitudinally compressed and the bowing members are radially expanded, the patch deployment mechanism being configured to radially retracted after the first therapeutic patch has been adhered to the first target portion of tissue, from a deployed configuration to the insertion configuration to separate the first therapeutic patch from the patch deployment mechanism leaving the first therapeutic patch in a desired position adhered to the first target portion of tissue.

14. The device of claim 13, further comprising:
a control wire coupled to the distal member of the patch deployment mechanism, movement of the control wire proximally relative to the patch deployment mechanism compressing the patch deployment mechanism along a longitudinal axis thereof and expanding the bowing members radially away from the longitudinal axis.

15. The device of claim 14, further comprising:
a tube slidably receiving the control wire, wherein the tube is coupled to the proximal member of the patch deployment mechanism so that movement of the control wire within the tube moves the distal member of the patch deployment mechanism relative to the distal member of the patch deployment mechanism to move the patch deployment mechanism between the insertion configuration and a radially expanded configuration.

16. The device of claim 15, further comprising:
a handle including a slider coupled to the control wire, the slider being configured so that movement of the slider relative the tube moves the control wire through the tube to move the patch deployment mechanism between the insertion configuration and the radially expanded configuration.

17. The device of claim 16, wherein the handle further includes a handle body and a spool slidably mounted on the handle body, the spool being coupled to the tube so that movement of the spool relative to the handle body moves the patch deployment mechanism relative to the cap.

18. The device of claim 13, wherein each of the bowing members is coupled to a distal surface of the proximal member and wherein each of the bowing members is coupled to a proximal surface of the distal member, the bowing members being arranged, in the insertion configuration, in a cylinder extending about a longitudinal axis of the patch deployment mechanism.

19. The device of claim 18, wherein the bowing members are coupled to the proximal and distal members radially within radially outer surfaces of the proximal and distal members with respect to the longitudinal axis of the patch deployment mechanism and wherein a distance between the radially outer surfaces of the bowing members and the radially outer surfaces of the proximal and distal members is selected to provide a clearance between the bowing members and the radially outer surfaces of the proximal and distal members to accommodate the first therapeutic patch.

20. The device of claim 19, wherein the proximal and distal members are substantially circular and are parallel to one another and centered about the longitudinal axis of the patch deployment mechanism to form a cylinder and wherein the cap is cylindrical and sized to slidably receive the patch deployment mechanism therein when the patch deployment mechanism is in the insertion configuration and wherein, in a radially expanded configuration, a maximum diameter defined by the bowing members is greater than an outer diameter of the cap.

21. The device of claim 13, wherein the bowing members are formed of one of Nitinol, Nitional cobalt alloy wire, pebax, nylon, ptfe, stainless steel, hypotube and a biocompatible hard plastic.

22. The device of claim 13, further comprising:
a second therapeutic patch wrapped around the patch deployment mechanism and radially outside the first therapeutic patch, the second therapeutic patch being configured to be forced radially outward into contact with and adhere to a second target portion of tissue when the patch deployment mechanism is moved to a radially expanded configuration, the patch deployment mechanism being configured to be moved, after the second therapeutic patch has been adhered to the second target portion of tissue as desired, from the radially expanded configuration to the insertion configuration to separate the second therapeutic patch from the patch deployment mechanism leaving the second therapeutic patch in a desired position adhered to the second target portion of tissue.

23. The device of claim 13, wherein the first therapeutic patch has a width extending along a longitudinal axis of the patch deployment mechanism and a length transverse to the width, the first therapeutic patch being wrapped around the patch deployment mechanism so that the length of the first therapeutic patch extends circumferentially around the patch deployment mechanism, the length of the first therapeutic patch being configured to be at least as great as a diameter of the patch deployment mechanism in the deployed configuration.

24. The device of claim 13, wherein the first therapeutic patch includes an adhesive having a predetermined cure time and wherein the first therapeutic patch is configured to be removable from tissue against which it has been pressed before the predetermined cure time has elapsed since the first therapeutic patch was pressed against the tissue.

25. A method for treating tissue, comprising:
inserting into a living body a flexible endoscope a device including a cap mounted on a distal end thereof, the device including, slidably received within the cap, a patch deployment mechanism with a first therapeutic patch wrapped therearound, the patch deployment mechanism being maintained during insertion in an insertion configuration in which the patch deployment mechanism and the first therapeutic patch are maintained within the cap;
moving the patch deployment mechanism distally out of the cap to expose the patch deployment mechanism and the first therapeutic patch;
expanding the patch deployment mechanism to move the first therapeutic patch radially outward into contact with a first target portion of tissue to be treated; and
radially contracting the patch deployment mechanism out of contact with the first therapeutic patch to leave the first therapeutic patch in a desired position adhered to the first target portion of tissue.

26. The method of claim 25, wherein the first therapeutic patch has a width extending along a longitudinal axis of the patch deployment mechanism and a length transverse to the width, the first therapeutic patch being wrapped around the patch deployment mechanism so that the length of the first therapeutic patch extends circumferentially around the patch deployment mechanism, the length of the first therapeutic patch being configured to be at least as great as a diameter of the patch deployment mechanism in a deployed configuration, further comprising the step of:
pressing the first therapeutic patch against the first target portion of tissue and moving the patch deployment mechanism relative to the first target portion of tissue in a direction corresponding to the length of the first therapeutic patch to unroll the first therapeutic patch from the patch deployment mechanism over the first target portion of tissue until the entire length of the first therapeutic patch has been adhered to the first target portion of tissue.

27. The method of claim 25, wherein the device further includes a second a therapeutic patch wrapped around the first therapeutic patch and the patch deployment mechanism.

28. The method of claim 25, wherein the patch deployment mechanism includes a plurality of bowing members which, in the insertion configuration form a cylinder having a diameter less than an inner diameter of the cap, further comprising, longitudinally compressing the patch deployment mechanism to radially expand the bowing members to push the first therapeutic patch radially outward.

29. The method of claim 28, wherein the device includes a distal member of the patch deployment mechanism coupled to a distal end of a control wire that extends to a proximal end that remains outside the living body and a tube coupled to a proximal member of the patch deployment mechanism, further comprising moving the control wire proximally relative to the tube to move the distal member proximally toward to the proximal member to longitudinally compress and radially expand the bowing members.
